(19) United States
(12) Patent Application Publication
(10) Pub. No.: US 2026/0235757 A1
(43) Pub. Date: Aug. 13, 2026

(54) OPTICAL MEASUREMENT DEVICE AND OPTICAL MEASUREMENT METHOD

(71) Applicant: SHIMADZU CORPORATION, Kyoto-shi, Kyoto (JP)

(72) Inventors: Takahide HATAHORI, Kyoto-shi, Kyoto (JP); Kenji TAKUBO, Kyoto-shi, Kyoto (JP)

(73) Assignee: SHIMADZU CORPORATION, Kyoto-shi, Kyoto (JP)

(21) Appl. No.: 19/155,162

(22) PCT Filed: Feb. 19, 2024
(86) PCT No.: PCT/JP2024/005689

§ 371 Date: Aug. 8, 2025

(30) Foreign Application Priority Data
Feb. 20, 2023 (JP) 2023-024079

(51) Int. Cl. G01S 17/86; G01S 7/484; G01S 7/4861; G01S 7/4865; G01S 17/89

(52) U.S. Cl. CPC G01S 17/86; G01S 7/484; G01S 7/4861; G01S 7/4865; G01S 17/89

(57) ABSTRACT

The present disclosure provides an optical measurement device comprising a laser source, an ultrasound source, a camera, a control circuit, and a data analysis unit.

### TECHNICAL FIELD

The present disclosure relates to an optical measurement device and an optical measurement method for measuring light modulated by ultrasound.

### BACKGROUND ART

As a method for minimally invasively measuring tissue within a living body, many techniques using light have been developed. For example, by irradiating light from outside the body and measuring the light that propagates through the living body and is emitted, biological information such as morphological information and metabolic information (e.g., oxygen saturation in the blood) of the tissue within the living body can be obtained. However, since tissue within a living body is a light-scattering medium, light irradiated from outside the body is diffused by the tissue, resulting in poor spatial resolution and the inability to measure deep parts. Therefore, an optical measurement device using ultrasound-modulated optical tomography (UOT), which combines light with ultrasound that propagates with low scattering within a living body, has been developed (Non-Patent Literatures 1 and 2). In UOT, biological information is obtained by measuring light modulated by ultrasound.

Non-Patent Literature 3 discloses acquiring information of a local region indicated by ultrasound by capturing a speckle pattern with and without irradiation of focused pulsed ultrasound using a CCD camera, and determining the change in the speckle pattern based on the difference thereof.

### PRIOR ART DOCUMENTS

### Non-Patent Literature

NON-PATENT LITERATURE 1: Wang, L.V.; Ku, G.; “Frequency-swept ultrasound-modulated optical tomography of scattering media,” Optics Letters 23(12) 975-977 (1998)

NON-PATENT LITERATURE 2: Elson, Daniel S., et al. “Ultrasound-mediated optical tomography: a review of current methods.” Interface Focus 1.4(2011 ):632-648.

NON-PATENT LITERATURE 3: Sasakura, Y.; Hissaka, M. “Reflection-type ultrasound-modulated speckle optical measurement method.” Japanese Journal of Medical Electronics and Biological Engineering, 2007, 45.4:235-241.

### SUMMARY OF THE INVENTION

### Technical Problem

However, when measuring tissue within a living body, the autocorrelation of the measured speckle pattern is lost (referred to as decorrelation) due to random state changes of the tissue caused by biological activities (biological fluctuation). As in Non-Patent Literature 3, when speckle patterns are captured both with and without ultrasound irradiation and the difference is taken, the output of the difference is affected by decorrelation.

Although using an image sensor with a short frame time can suppress the influence of decorrelation, such image sensors are problematic in that they are relatively expensive.

The present disclosure has been made to solve such problems, and an object thereof is to suppress the influence of biological fluctuation on a measured speckle pattern in an optical measurement device and an optical measurement method using ultrasound-modulated optical tomography.

### Solution to Problem

An optical measurement device of the present disclosure includes: a light source that irradiates pulsed laser light into a living body; an ultrasound source that irradiates ultrasound to a measurement position at a predetermined depth within the living body; an image sensor that detects the laser light that has passed through a region within the living body including the measurement position; a control circuit that controls irradiation timing for irradiating the laser light from the light source; and an arithmetic circuit that extracts a signal component modulated by the ultrasound from the laser light detected by the image sensor. The control circuit irradiates a first laser light and a second laser light at a time interval shorter than one frame time of the image sensor, and controls the irradiation timing such that an irradiation time of the first laser light and an irradiation time of the second laser light respectively correspond to different exposure times of consecutive frames. The ultrasound source irradiates the ultrasound such that the ultrasound reaches the measurement position at the irradiation time of the first laser light or the second laser light. The arithmetic circuit extracts the signal component modulated by the ultrasound based on a detection signal of the first laser light and a detection signal of the second laser light detected by the image sensor.

An optical measurement method of the present disclosure is an optical measurement method in an optical measurement device including: a light source that irradiates pulsed laser light into a living body; an ultrasound source that irradiates ultrasound to a measurement position at a predetermined depth within the living body; an image sensor that detects the laser light that has passed through a region within the living body including the measurement position; a control circuit that controls irradiation timing for irradiating the laser light from the light source; and an arithmetic circuit that extracts a signal component modulated by the ultrasound from the laser light detected by the image sensor. The optical measurement method includes: a step of irradiating a first laser light and a second laser light at a time interval shorter than one frame time of the image sensor, and controlling the irradiation timing such that an irradiation time of the first laser light and an irradiation time of the second laser light respectively correspond to different exposure times of consecutive frames; a step of the ultrasound source irradiating the ultrasound such that the ultrasound reaches the measurement position at the irradiation time of the first laser light or the second laser light; and a step of extracting the signal component modulated by the ultrasound based on a detection signal of the first laser light and a detection signal of the second laser light detected by the image sensor.

### Advantageous Effects of Invention

In the optical measurement device and the optical measurement method of the present disclosure, the first laser light and the second laser light are irradiated at a time interval shorter than one frame time of the image sensor, and a first exposure time during which the first laser light is irradiated and a second exposure time during which the second laser light is irradiated are different exposure times of consecutive frames. Therefore, the measurement by the image sensor can be completed within a decorrelation time, which reduces the influence of decorrelation and improves measurement accuracy.

### BRIEF DESCRIPTION OF THE DRAWINGS

FIG. 1 is a schematic diagram of an optical measurement device according to an embodiment.

FIG. 2 is a flowchart showing an optical measurement method according to the embodiment.

FIG. 3 is a timing chart showing a pulse laser lighting timing of the optical measurement device according to the embodiment.

FIG. 4 is a schematic diagram showing a pulse laser lighting mode of the optical measurement device according to the embodiment.

FIG. 5 is a timing chart showing another pulse laser lighting timing of the optical measurement device according to the embodiment.

FIG. 6 is a schematic diagram of a measurement system of a modified example using the optical measurement device according to the embodiment.

FIG. 7 is a block diagram of the measurement system of the modified example.

### DESCRIPTION OF EMBODIMENTS

Hereinafter, embodiments will be described in detail with reference to the drawings. Note that the same or corresponding parts in the drawings are denoted by the same reference numerals, and description thereof will not be repeated.

### Optical Measurement Device

Hereinafter, an optical measurement device that measures light modulated by ultrasound to measure tissue within a living body in a minimally invasive manner will be described. The optical measurement device according to the present embodiment can be applied, for example, to an optical measurement device that optically measures brain activity of a subject in a minimally invasive manner by near-infrared spectroscopy (NIRS). Of course, the optical measurement device according to the present embodiment can also be applied to a measurement device that measures oxygen saturation in blood, in addition to an optical measurement device that measures brain activity by near-infrared spectroscopy.

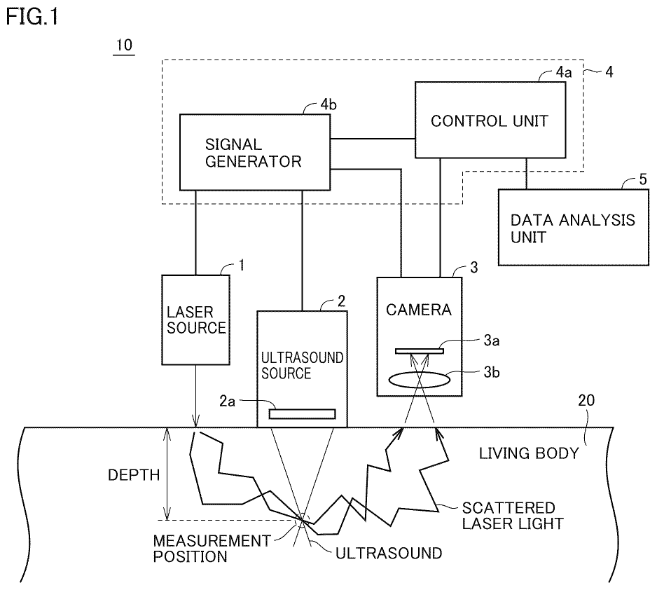

FIG. 1 is a schematic diagram of an optical measurement device 10 according to an embodiment. The optical measurement device 10 is composed of a laser source 1, an ultrasound source 2, a camera 3, a control circuit 4, and a data analysis unit 5. Note that the control circuit 4 and the data analysis unit 5 can be configured on a single computer (not shown), and external devices such as a memory or a printer can be connected as necessary. Further, the laser source 1, the ultrasound source 2, the camera 3, and the like can be integrally configured, and these and the control circuit 4 may be integrally configured to be wearable by a subject.

The laser source 1 is a light source that irradiates laser light into a living body 20, and is, for example, a semiconductor laser element. In the present embodiment, the laser source 1 is controlled to generate pulsed laser light (pulse laser light). The laser source 1 irradiates laser light in a near-infrared light wavelength region with high transmittance for the living body 20 (for example, 780 nm, etc.). As shown in FIG. 1, the laser light irradiated from the laser source 1 to the living body 20 is scattered by the tissue within the living body 20 and reaches a measurement position. Further, the laser source 1 can irradiate pulsed laser light of about several tens of nanoseconds to several nanoseconds.

The ultrasound source 2 is an ultrasound generator that irradiates ultrasound to a measurement position at a predetermined depth within the living body 20. The ultrasound source 2 is provided with a focusing unit 2a for focusing the irradiated ultrasound at the measurement position within the living body 20. Here, speckle fluctuation increases as the sound pressure of the ultrasound increases. Therefore, by focusing the ultrasound at the measurement position within the living body 20 with the focusing unit 2a, a region of high sound pressure is limited to the measurement position, thereby reducing speckle fluctuation that occurs in regions where ultrasound exists other than the measurement position. The ultrasound irradiated from the ultrasound source 2 may be continuous ultrasound or pulsed ultrasound. However, by using pulsed ultrasound as the ultrasound irradiated from the ultrasound source 2, the length of the ultrasound in the traveling direction can be shortened, and the region where the ultrasound exists can be limited to the measurement position. Further, when irradiating pulsed ultrasound from the ultrasound source 2, a time (delay time) is required for the pulsed ultrasound irradiated from the surface of the living body 20 to reach the measurement position. Therefore, it is necessary to control the timing (second timing) at which the ultrasound source 2 irradiates the ultrasound so that the pulsed ultrasound reaches the measurement position in synchronization with the time at which the pulsed laser light irradiated from the laser source 1 reaches the measurement position. Note that since the distance to the measurement position is sufficiently short with respect to the speed of light, the time at which the pulsed laser light irradiated from the laser source 1 reaches the measurement position is substantially the same as the irradiation time at which the laser source 1 irradiated the pulsed laser light.

The camera 3 includes an image sensor 3a for detecting laser light from the measurement position, and a lens 3b for forming an image on the image sensor 3a. In the optical measurement device 10, since it is necessary to capture a speckle pattern, an image sensor 3a such as a Charge Coupled Device (CCD) sensor or a Complementary Metal-Oxide-Semiconductor (CMOS) sensor, which is a multi-element photodetector, is used instead of a single-element photodetector like a photomultiplier tube. By adopting a CCD sensor or a CMOS sensor for the image sensor 3a, the manufacturing cost of the optical measurement device 10 can be reduced. Note that a speckle pattern is an aggregate of speckle grains (spot-like particles) generated by multiple interference of light that has been multiply scattered inside the tissue of the living body 20. Therefore, it is preferable that the pixel size of the image sensor 3a is smaller than the average size of the speckle grains to be captured.

An image sensor 3a such as a CCD sensor generally has a frame rate of several tens of fps to several hundreds of fps. In contrast, the frequency of ultrasound is as high as several MHz, and the image sensor 3a cannot follow the change of the ultrasound. Therefore, the optical measurement device 10 uses a stroboscopic method to irradiate pulsed laser light to the tissue in the living body 20 that is being irradiated with ultrasound, and captures the speckle pattern with the image sensor 3a in an exposure state. Furthermore, in the optical measurement device 10, the irradiation timing (first timing) at which the laser source 1 irradiates the laser light is controlled so that the measurement can be completed within a decorrelation time so that the autocorrelation of the speckle pattern measured is not lost due to biological fluctuation.

The control circuit 4 controls a first timing for irradiating the laser light from the laser source 1 and a second timing for irradiating the ultrasound from the ultrasound source 2. Specifically, the control circuit 4 has a control unit 4a and a signal generator 4b. The control unit 4a sets a first timing for irradiating a first laser light and a second laser light at a time interval shorter than one frame time of the image sensor 3a to different exposure times of consecutive frames, based on the frame rate of the image sensor 3a. Further, the control unit 4a sets a second timing so that the ultrasound reaches the measurement position at the irradiation time of the second laser light. Note that, in the present disclosure, it is described that the control unit 4a controls the timing (second timing) at which the ultrasound source 2 irradiates the ultrasound, but instead of the control unit 4a, the ultrasound source 2 may control the timing at which the ultrasound is irradiated so that the ultrasound reaches the measurement position at the irradiation time of the first laser light or the second laser light. Here, since the laser light is pulsed laser light, it is a laser light having a pulse width. Therefore, the irradiation time of the laser light is defined as, for example, the rising edge time of the pulse. Of course, the irradiation time of the laser light may be defined as, for example, the time of the median value of the pulse, or the falling edge time of the pulse.

The signal generator 4b supplies a drive signal to the laser source 1 so as to irradiate the first laser light and the second laser light at the first timing set by the control unit 4a. Further, the signal generator 4b supplies a drive signal to the ultrasound source 2 so as to irradiate the ultrasound at the second timing set by the control unit 4a.

The data analysis unit 5 is an arithmetic circuit that extracts a signal component modulated by the ultrasound from the laser light detected by the camera 3. Specifically, the data analysis unit 5 extracts a signal component (speckle pattern) modulated by the ultrasound based on the detection signal of the first laser light and the detection signal of the second laser light detected by the camera 3. Specifically, in the data analysis unit 5, a first speckle pattern not modulated by ultrasound is obtained from the detection signal of the first laser light, and a second speckle pattern modulated by ultrasound is obtained from the detection signal of the second laser light. The first speckle pattern and the second speckle pattern are speckle patterns captured within a decorrelation time during which biological fluctuation does not occur. Therefore, the data analysis unit 5 can obtain a speckle pattern with a high S/N ratio by calculating the difference value between the first speckle pattern and the second speckle pattern.

### Optical Measurement Method

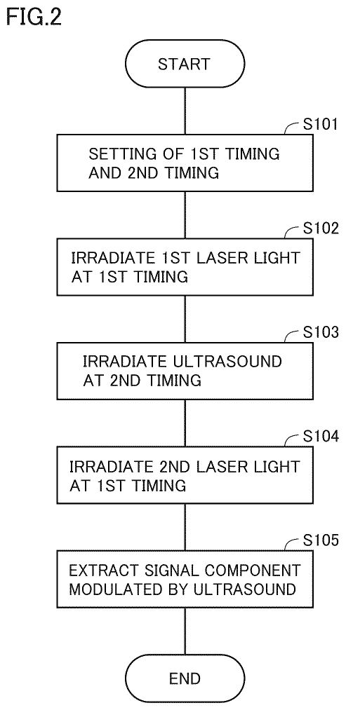

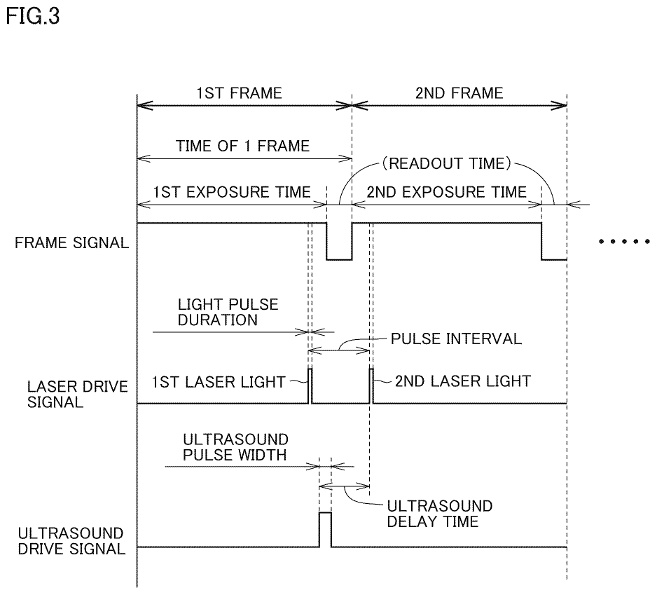

A method for obtaining a speckle pattern modulated by ultrasound using the optical measurement device 10 will be described. FIG. 2 is a flowchart showing an optical measurement method according to the embodiment. FIG. 3 is a timing chart showing a pulse laser lighting timing of the optical measurement device 10 according to the embodiment.

First, the optical measurement device 10 sets a first timing for irradiating the laser light from the laser source 1 and a second timing for irradiating the ultrasound from the ultrasound source 2 in the control unit 4a (Step S101). First, the first timing is a lighting timing of a pulse laser for realizing optical measurement by UOT with the camera 3 using the image sensor 3a within the decorrelation time. Specifically, the first timing is a timing for causing the laser source 1 to irradiate two pulse laser lights, a first laser light and a second laser light, at a time interval shorter than one frame time of the image sensor 3a, at the timing of switching frames of the image sensor 3a, as shown in FIG. 3. Further, in the first timing, the irradiation time of the first laser light and the irradiation time of the second laser light respectively correspond to different exposure times of consecutive frames. Note that the irradiation time of the first laser light corresponds to the exposure time of the first frame (first exposure time), and the irradiation time of the second laser light corresponds to the exposure time of the second frame (second exposure time).

Here, one frame time of the image sensor 3a includes an exposure time and a readout time for the data captured during the exposure time. The period when the frame signal is in the ON state is the exposure time, and the period when the frame signal is in the OFF state is the readout time. Further, the time interval at which the first laser light and the second laser light are irradiated is also referred to as a pulse interval. This pulse interval may be equal to or less than the decorrelation time. It is known that the decorrelation time is generally less than 1 ms, and it is preferable to obtain an average value of the decorrelation times of a plurality of subjects and set the pulse interval to, for example, 500 μs or less.

In the optical measurement device 10, as will be described later, the control unit 4a controls the irradiation timing of the laser light so that the timings of irradiating the laser light become the exposure times of different frames. Specifically, the control unit 4a synchronizes the laser source 1, the ultrasound source 2, and the camera 3 in advance with a signal (for example, a synchronization signal, etc.) output from the signal generator 4b, and controls the laser source 1 to irradiate the laser light at a timing that results in different frame exposure times. Of course, in addition to synchronously controlling the laser source 1, the ultrasound source 2, and the camera 3, the control unit 4a may determine that the frame exposure times are different and control the irradiation timing of the laser light. For example, the control unit 4a determines the different frame exposure times based on a signal for each frame (for example, a trigger signal, etc.) output from the camera 3, and controls the irradiation timing of the laser light.

The period during which the first laser light and the second laser light are ON (the period when the laser drive signal is in the ON state) is also referred to as a light pulse duration. By making the light pulse duration shorter than the period of the ultrasound (for example, ⅛ period of the ultrasound or less), the optical measurement device 10 can perform measurement in a time width in which the speckle fluctuation caused by the ultrasound can be regarded as stationary. Therefore, the optical measurement device 10 can obtain a direct difference value between a speckle pattern not modulated by ultrasound and a speckle pattern modulated by ultrasound in a certain state, enabling measurement with a high S/N ratio. However, if the light pulse duration is made shorter than the period of the ultrasound, the amount of light that can be detected by the image sensor 3a decreases. In order to obtain a sufficient amount of light that can be detected by the image sensor 3a, it is preferable to configure the first laser light and the second laser light to include a plurality of pulsed laser lights.

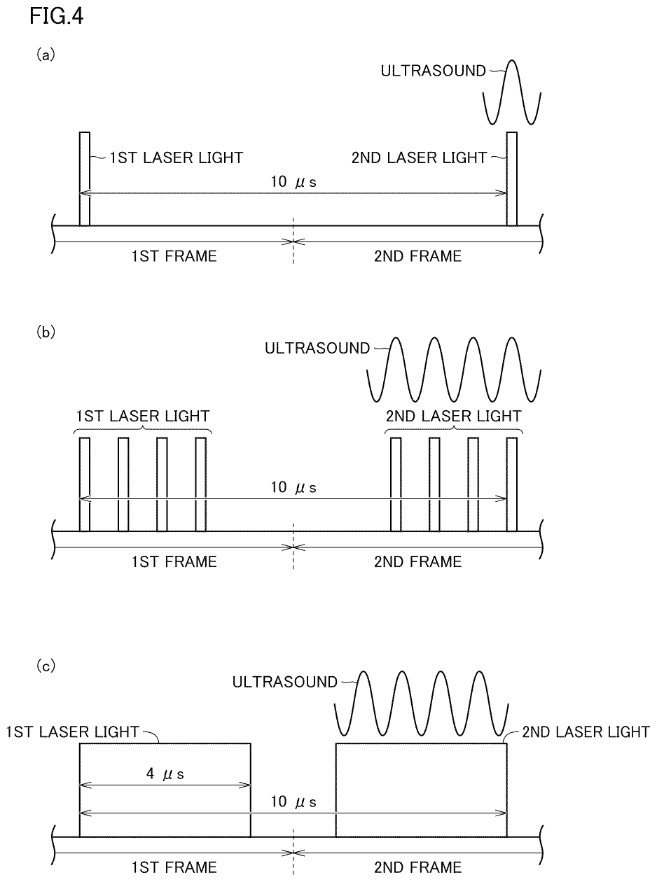

FIG. 4 is a schematic diagram showing a pulse laser lighting mode of the optical measurement device 10 according to the embodiment. In FIG. 4(a), a first laser light including one pulse laser light is irradiated in the first frame, and a second laser light including one pulse laser light is irradiated in the second frame. The time interval (pulse interval) between the first laser light and the second laser light is 10 μs. The light pulse duration of the first laser light and the second laser light is shorter than the period of the ultrasound as shown in FIG. 4(a). The first laser light and the second laser light shown in FIG. 3 adopt the pulse laser lighting mode shown in FIG. 4(a), but the pulse laser lighting modes shown in FIG. 4(b) and FIG. 4(c) described below may also be adopted.

In FIG. 4(b), a first laser light including four pulse laser lights is irradiated in the first frame, and a second laser light including four pulse laser lights is irradiated in the second frame. The time interval (pulse interval) from the first pulse laser light of the first laser light to the fourth pulse laser light of the second laser light is 10 μs. The respective light pulse durations of one pulse laser light of the first laser light and one pulse laser light of the second laser light are shorter than the period of the ultrasound as shown in FIG. 4(b). However, the total light pulse duration of the four pulse laser lights is four times the light pulse duration of FIG. 4(a), so the amount of light that can be detected by the image sensor 3a increases. However, since the four pulse laser lights are lit so as to be at the same peak position of each ultrasound wave, a speckle pattern modulated by the portion of the ultrasound at the same peak position is obtained, and optical measurement not affected by the speckle pattern modulated by the portions of the ultrasound other than the peak is possible. Note that the number of pulse laser lights included in the first laser light and the second laser light is not limited to four, and may be a plurality.

In FIG. 4(c), a first laser light including one pulse laser light is irradiated in the first frame, and a second laser light including one pulse laser light is irradiated in the second frame. The time interval (pulse interval) between the first laser light and the second laser light is 10 μs. However, the light pulse duration of the first laser light and the second laser light is 4 μs, which corresponds to the length of four periods of the ultrasound, as shown in FIG. 4(c). Since the light pulse duration becomes the length of four periods of the ultrasound, the amount of light that can be detected by the image sensor 3a increases. However, since the speckle patterns modulated by the ultrasound for four periods are superimposed on the first laser light and the second laser light, a time average of the speckle patterns modulated by the ultrasound for four periods is obtained.

Returning to FIG. 3, the second timing is a timing for causing the ultrasound source 2 to irradiate ultrasound so that the ultrasound reaches the measurement position at the irradiation time of the second laser light. Specifically, as shown in FIG. 3, the second timing is a timing for causing the ultrasound source 2 to irradiate pulsed ultrasound before the irradiation time of the second laser light by an ultrasound delay time. Note that the ultrasound delay time can be calculated from the distance from the ultrasound source 2 to the measurement position. Further, since the ultrasound irradiated by the ultrasound source 2 is pulsed ultrasound, the period during which the ultrasound is irradiated (the period when the ultrasound drive signal is in the ON state) is also referred to as an ultrasound pulse width. Of course, the ultrasound irradiated by the ultrasound source 2 is not limited to pulsed ultrasound, and may be continuous ultrasound.

Furthermore, in FIG. 3, it has been described that the ultrasound is irradiated in synchronization with the time when the second laser light irradiates the measurement position, but the ultrasound may be irradiated in synchronization with the time when the first laser light irradiates the measurement position. When irradiating the ultrasound in synchronization with the time when the first laser light irradiates the measurement position, the second timing is a timing for causing the ultrasound source 2 to irradiate the ultrasound so that the ultrasound reaches the measurement position at the irradiation time of the first laser light. The ultrasound source 2 may irradiate the ultrasound so that the ultrasound reaches the measurement position in synchronization with either the irradiation time of the first laser light or the irradiation time of the second laser light.

Returning to FIG. 2, the optical measurement device 10 supplies a laser drive signal from the signal generator 4b to the laser source 1 based on the first timing set by the control unit 4a, and causes the laser source 1 to irradiate the first laser light (Step S102).

Next, the optical measurement device 10 supplies an ultrasound drive signal from the signal generator 4b to the ultrasound source 2 based on the second timing set by the control unit 4a, and causes the ultrasound source 2 to irradiate the ultrasound (Step S103).

Next, the optical measurement device 10 supplies a laser drive signal from the signal generator 4b to the laser source 1 based on the first timing set by the control unit 4a, and causes the laser source 1 to irradiate the second laser light (Step S104).

Next, in the optical measurement device 10, the data analysis unit 5 extracts a signal component modulated by the ultrasound from the detected speckle patterns of the two frames (Step S105). The data analysis unit 5 obtains a first speckle pattern not modulated by ultrasound from the detection signal of the first laser light, and a second speckle pattern modulated by ultrasound from the detection signal of the second laser light. The data analysis unit 5 can extract a signal component (speckle pattern) with a high S/N ratio by calculating the difference value between the first speckle pattern and the second speckle pattern.

### Modified Example of Lighting Timing

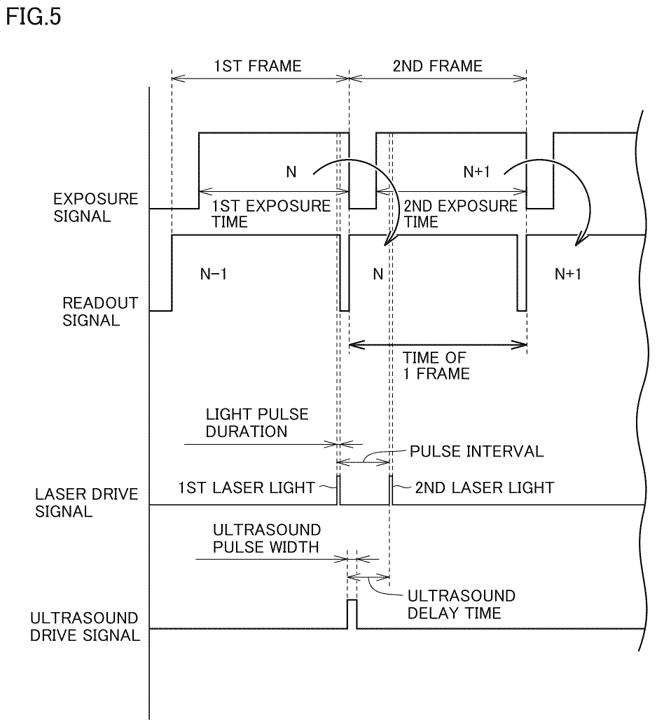

One frame time of the image sensor 3a shown in FIG. 3 includes an exposure time and a readout time for the data captured during the exposure time. However, the configuration of one frame of the image sensor differs depending on the type of sensor, the readout method, and the like, and is not limited to the configuration shown in FIG. 3. FIG. 5 is a timing chart showing another pulse laser lighting timing of the optical measurement device 10 according to the embodiment. One frame time of the image sensor shown in FIG. 5 includes an exposure time and a readout time for the data captured in the frame prior to the exposure time. In the image sensor shown in FIG. 5, the data captured during the first exposure time of the first frame is read out during the readout time of the second frame. Therefore, in the second frame, the second exposure time of the second frame and the readout time for reading out the data of the first frame occur in the same time period.

Also in the first timing shown in FIG. 5, two pulse laser lights, a first laser light and a second laser light, are irradiated at a time interval shorter than one frame time of the image sensor. Further, also in the first timing shown in FIG. 5, the first exposure time during which the first laser light is irradiated and the second exposure time during which the second laser light is irradiated are exposure times of different frames. The first exposure time is the exposure time of the first frame, and the second exposure time is the exposure time of the second frame.

In FIG. 5, the period when the exposure signal is in the ON state is the exposure time, and the period when the readout signal is in the ON state is the readout time. Further, also in the first laser light and the second laser light shown in FIG. 5, the pulse interval is equal to or less than the decorrelation time, and the light pulse duration is made shorter than the period of the ultrasound.

### Application to Brain Function Measurement Device

The optical measurement device 10 according to the present embodiment can be applied to a measurement device for measuring brain function, and can measure brain activity of a subject in a minimally invasive manner by near-infrared spectroscopy. When applying the optical measurement device 10 to a measurement device for measuring brain function, the laser source 1, the ultrasound source 2, and the camera 3 shown in FIG. 1 are arranged on the head surface of the subject. That is, the living body 20 shown in FIG. 1 is the head surface of the subject. This allows the optical measurement device 10 to measure the brain activity of the subject in a minimally invasive manner, and to visualize the activity state near the brain surface in real time by functional near-infrared spectroscopy (fNIRS).

The laser source 1 is configured to irradiate the first laser light and the second laser light shown in FIG. 3 from the head surface of the subject to the measurement position. The laser source 1 includes, for example, a semiconductor laser, and is configured to be able to irradiate laser light of multiple wavelengths in a near-infrared light wavelength region with high biological transmittance (for example, light of three wavelengths of 780 nm, 805 nm, and 830 nm). The camera 3 includes an image sensor such as a CCD sensor, and detects the laser light from the measurement position. The camera 3 outputs an electric signal corresponding to the detected light. The ultrasound source 2 irradiates ultrasound to the measurement position in the head of the subject.

Then, the data analysis unit 5 analyzes the change in the amount of hemoglobin (oxyhemoglobin, deoxyhemoglobin, and total hemoglobin) associated with brain activity, based on the speckle pattern measured by the camera 3. This enables the optical measurement device 10 to acquire the change in the amount of hemoglobin associated with brain activity, that is, the change in blood flow and the activation state of oxygen metabolism, in a minimally invasive manner.

### MODIFIED EXAMPLE

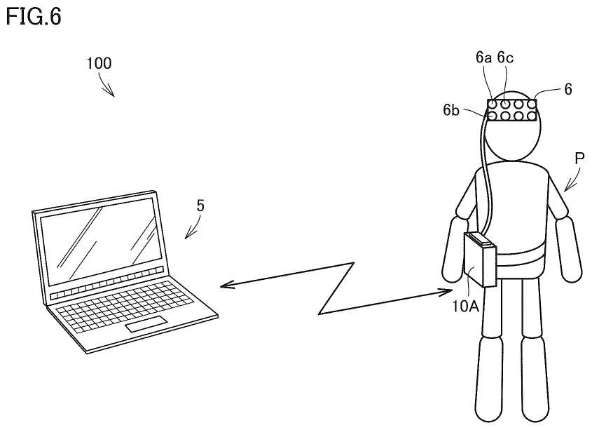

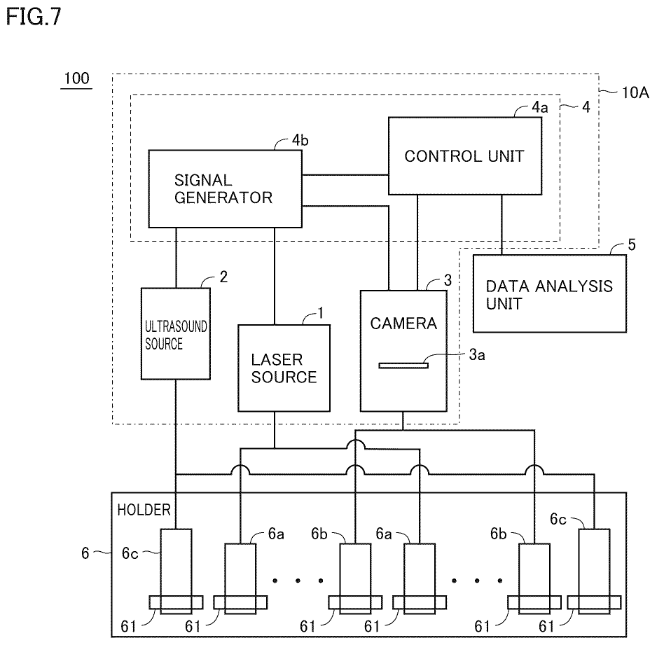

When applying the optical measurement device 10 to a measurement system for measuring brain function, measurement light may be irradiated to the head of the subject from a plurality of measurement probes placed on the head of the subject, and the measurement light scattered within the brain in the head of the subject may be received. FIG. 6 is a schematic diagram of a measurement system 100 of a modified example using the optical measurement device according to the embodiment. FIG. 7 is a block diagram of the measurement system 100 of the modified example.

As shown in FIG. 6, the measurement system 100 for measuring brain function can measure the brain activity of a subject Pin a minimally invasive manner, and is configured as a brain function imaging device that visualizes the activity state near the brain surface in real time by functional near-infrared spectroscopy. Further, the measurement system 100 is composed of, for example, a main body unit 10A worn by the subject P, and a data analysis unit 5 which is a computer that receives and analyzes the data measured by the main body unit 10A by wireless communication.

Thereby, in the measurement system 100, the subject P is not restricted to the vicinity of the data analysis unit 5 even during brain function measurement, and the subject P can carry the main body unit 10A and move freely, making it possible to perform brain function measurement in an environment closer to daily life.

The main body unit 10A is composed of the laser source 1, the ultrasound source 2, the camera 3, and the control circuit 4. Further, the measurement system 100 includes a holder 6 that is worn on the head of the subject P and includes a plurality of attachment parts 61 for attaching a light-transmitting probe 6a, a light-receiving probe 6b, and an ultrasound probe 6c. Then, the light-transmitting probe 6a, the light-receiving probe 6b, and the ultrasound probe 6c are respectively attached to the holder 6 worn on the head of the subject P, thereby being arranged on the head surface of the subject P.

The light-transmitting probe 6a is connected to the laser source 1 via an optical fiber, and irradiates the first laser light and the second laser light shown in FIG. 3 from the head surface of the subject P to the measurement position. The light-receiving probe 6b is connected to the camera 3 via an optical fiber, and detects the laser light from the measurement position. The ultrasound probe 6c is connected to the ultrasound source 2 via wiring, and irradiates ultrasound from the head surface of the subject P to the measurement position.

The laser source 1 is configured to irradiate the first laser light and the second laser light to the head of the subject P from the light-transmitting probe 6a via an optical fiber. The laser source 1 includes, for example, a semiconductor laser, and is configured to be able to irradiate laser light of multiple wavelengths in a near-infrared light wavelength region with high biological transmittance. The camera 3 includes an image sensor such as a CCD sensor, and is configured to acquire and detect the light incident on the light-receiving probe 6b via an optical fiber. The camera 3 outputs an electric signal corresponding to the detected light. The ultrasound source 2 irradiates ultrasound from the ultrasound probe 6c to the measurement position in the head of the subject P.

Then, the data analysis unit 5 is configured to analyze the change in the amount of hemoglobin associated with brain activity, based on the speckle pattern measured by the camera 3. This enables the measurement system 100 to acquire the change in the amount of hemoglobin associated with brain activity, that is, the change in blood flow and the activation state of oxygen metabolism, in a minimally invasive manner. Further, in the measurement system 100, brain activity is measured for each measurement point (measurement channel) constituted by the light-transmitting probe 6a, the light-receiving probe 6b, and the ultrasound probe 6c, and is configured to acquire a two-dimensional distribution.

In the measurement system 100, the ultrasound probe 6c may further be an ultrasound phased array to enable steering and scanning of the ultrasound. Further, in the measurement system 100, a bundle fiber may be used for the light-receiving optical fiber that connects the light-receiving probe 6b and the camera 3.

### Aspects

It is understood by those skilled in the art that the embodiments described above are specific examples of the following aspects.

### (Item 1)

An optical measurement device according to one aspect includes: a light source that irradiates pulsed laser light into a living body; an ultrasound source that irradiates ultrasound to a measurement position at a predetermined depth within the living body; an image sensor that detects the laser light that has passed through a region within the living body including the measurement position; a control circuit that controls irradiation timing for irradiating the laser light from the light source; and an arithmetic circuit that extracts a signal component modulated by the ultrasound from the laser light detected by the image sensor. The control circuit irradiates a first laser light and a second laser light at a time interval shorter than one frame time of the image sensor, and controls the irradiation timing such that an irradiation time of the first laser light and an irradiation time of the second laser light respectively correspond to different exposure times of consecutive frames. The ultrasound source irradiates the ultrasound such that the ultrasound reaches the measurement position at the irradiation time of the first laser light or the second laser light. The arithmetic circuit extracts the signal component modulated by the ultrasound based on a detection signal of the first laser light and a detection signal of the second laser light detected by the image sensor.

According to the optical measurement device described in item 1, the first laser light and the second laser light are irradiated at a time interval shorter than one frame time of the image sensor, and a first exposure time during which the first laser light is irradiated and a second exposure time during which the second laser light is irradiated are different exposure times of consecutive frames. Therefore, the measurement by the image sensor can be completed within a decorrelation time, which reduces the influence of decorrelation and improves measurement accuracy.

### (Item 2)

The optical measurement device according to item 1, wherein the first laser light and the second laser light are composed of a plurality of pulsed laser lights.

According to the optical measurement device described in item 2, since a plurality of pulsed laser lights are irradiated to the measurement position, the amount of laser light detected by the image sensor can be increased.

### (Item 3)

The optical measurement device according to item 1 or 2, wherein a light pulse duration of the first laser light and the second laser light is shorter than a period of the ultrasound.

According to the optical measurement device described in item 3, by making the light pulse duration shorter than the period of the ultrasound, the speckle fluctuation caused by the ultrasound can be regarded as stationary.

### (Item 4)

The optical measurement device according to any one of items 1 to 3, wherein the light source can irradiate laser light with different peak wavelengths.

According to the optical measurement device described in item 4, since the light source can irradiate laser light with different peak wavelengths, various measurements such as oxygen saturation in the blood become possible.

### (Item 5)

The optical measurement device according to any one of items 1 to 4, wherein the ultrasound source can irradiate pulsed ultrasound.

According to the optical measurement device described in item 5, since the ultrasound source irradiates pulsed ultrasound, speckle fluctuation caused by the ultrasound can be reduced.

### (Item 6)

The optical measurement device according to any one of items 1 to 5, wherein the ultrasound source irradiates the ultrasound such that the ultrasound is focused at the measurement position.

According to the optical measurement device described in item 6, since the ultrasound source irradiates the ultrasound such that the ultrasound is focused at the measurement position, speckle fluctuation caused by the ultrasound other than at the measurement position can be reduced.

### (Item 7)

The optical measurement device according to any one of items 1 to 6, wherein a time interval for irradiating the first laser light and the second laser light is 500 us or less.

According to the optical measurement device described in item 7, by setting the time interval for irradiating the first laser light and the second laser light to 500 us or less, measurement within the decorrelation time becomes easy.

### (Item 8)

The optical measurement device according to item 4, wherein the light source can irradiate laser light of multiple wavelengths in a near-infrared light wavelength region.

According to the optical measurement device described in item 8, since the light source irradiates laser light of multiple wavelengths in the near-infrared light wavelength region, it is possible to measure the oxygen saturation in the blood.

### (Item 9)

An optical measurement method in an optical measurement device including: a light source that irradiates pulsed laser light into a living body; an ultrasound source that irradiates ultrasound to a measurement position at a predetermined depth within the living body; an image sensor that detects the laser light that has passed through a region within the living body including the measurement position; a control circuit that controls irradiation timing for irradiating the laser light from the light source; and an arithmetic circuit that extracts a signal component modulated by the ultrasound from the laser light detected by the image sensor. The optical measurement method includes: a step of irradiating a first laser light and a second laser light at a time interval shorter than one frame time of the image sensor, and controlling the irradiation timing such that an irradiation time of the first laser light and an irradiation time of the second laser light respectively correspond to different exposure times of consecutive frames; a step of the ultrasound source irradiating the ultrasound such that the ultrasound reaches the measurement position at the irradiation time of the first laser light or the second laser light; and a step of extracting the signal component modulated by the ultrasound based on a detection signal of the first laser light and a detection signal of the second laser light detected by the image sensor.

According to the optical measurement method described in item 9, the first laser light and the second laser light are irradiated at a time interval shorter than one frame time of the image sensor, and a first exposure time during which the first laser light is irradiated and a second exposure time during which the second laser light is irradiated are different exposure times of consecutive frames. Therefore, the measurement by the image sensor can be completed within a decorrelation time, which reduces the influence of decorrelation and improves measurement accuracy.

The embodiments disclosed this time should be considered as illustrative in all respects and not restrictive. The scope of the present invention is indicated by the claims rather than by the description of the embodiments above, and is intended to include all modifications within the meaning and scope equivalent to the claims.

### REFERENCE SIGNS LIST

1 Laser source, 2 Ultrasound source, 2a Focusing unit, 3 Camera, 3a Image sensor, 3b Lens, 4 Control circuit, 4a Control unit, 4b Signal generator, 5 Data analysis unit, 6 Holder, 6a Light-transmitting probe, 6b Light-receiving probe, 6c Ultrasound probe, 10 Optical measurement device, 10A Main body unit, 20 Living body, 61 Attachment part, 100 Measurement system.

1. An optical measurement device, comprising: a light source configured to irradiate pulsed laser light into a living body; an ultrasound source configured to irradiate ultrasound to focus at a measurement position at a predetermined depth within the living body; an image sensor configured to detect the laser light that has passed through a region within the living body including the measurement position; a control circuit configured to control irradiation timing for irradiating the laser light from the light source; and an arithmetic circuit configured to extract a signal component modulated by the ultrasound from the laser light detected by the image sensor, wherein the control circuit is configured to: irradiate a first laser light and a second laser light at a time interval shorter than one frame time of the image sensor, and control the irradiation timing such that an irradiation time of the first laser light and an irradiation time of the second laser light respectively correspond to different exposure times of consecutive frames, wherein the ultrasound source is configured to irradiate the ultrasound such that the ultrasound reaches the measurement position at the irradiation time of the first laser light or the second laser light, and wherein the arithmetic circuit is configured to: extract the signal component modulated by the ultrasound based on a detection signal of the first laser light and a detection signal of the second laser light detected by the image sensor, the light source is capable of irradiating the laser light with different peak wavelengths, and a time interval for irradiating the first laser light and the second laser light is 500 μs or less.

2. The optical measurement device according to claim 1, wherein the first laser light and the second laser light are composed of a plurality of the pulsed laser lights, a light pulse duration of the first laser light and the second laser light is shorter than a period of the ultrasound, and the ultrasound source is capable of irradiating pulsed ultrasound.

3-7. (canceled)

8. The optical measurement device according to claim 1, wherein the light source is capable of irradiating the laser light of multiple wavelengths in a near-infrared light wavelength region.

9. An optical measurement method in an optical measurement device comprising a light source configured to irradiate pulsed laser light into a living body, an ultrasound source configured to irradiate ultrasound to focus at a measurement position at a predetermined depth within the living body, an image sensor configured to detect the laser light that has passed through a region within the living body including the measurement position, a control circuit configured to control irradiation timing for irradiating the laser light from the light source, and an arithmetic circuit configured to extract a signal component modulated by the ultrasound from the laser light detected by the image sensor, the optical measurement method comprising the steps of: controlling the irradiation timing to irradiate a first laser light and a second laser light at a time interval shorter than one frame time of the image sensor, such that an irradiation time of the first laser light and an irradiation time of the second laser light respectively correspond to different exposure times of consecutive frames; irradiating, by the ultrasound source, the ultrasound such that the ultrasound reaches the measurement position at the irradiation time of the first laser light or the second laser light; and extracting the signal component modulated by the ultrasound based on a detection signal of the first laser light and a detection signal of the second laser light detected by the image sensor, wherein the light source is capable of irradiating the laser light with different peak wavelengths, and a time interval for irradiating the first laser light and the second laser light is 500 μs or less.
